(19) United States
(12) Patent Application Publication
(10) Pub. No.: US 2005/0170401 A1
(43) Pub. Date: Aug. 4, 2005

(54) Hybridization apparatus and method

(75) Inventor: Takahiro Sugiyama, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/044,172

(22) Filed: Jan. 28, 2005

(30) Foreign Application Priority Data
Jan. 29, 2004 (JP) 2004-021683

(51) Int. Cl. C12Q 1/68; C12M 1/34

(52) U.S. Cl. 435/6; 435/287.2

(57) ABSTRACT

Hybridization between a sample nucleic acid and a probe nucleic acid with an assay having the probe nucleic acid bound on the surface of a carrier, in which a liquid is fed to the surface of the carrier and the state of the thus-fed liquid is detected. The liquid may, for example, be a hybridization liquid containing the ample nucleic acid, or it may be a washing liquid for flushing byproducts and/or reactants away from the carrier in preparation for detection of hybridization results. Because the state of the liquid is detected, accuracy of hybridization assays is improved, since it becomes possible to detect and avoid difficulties encountered during hybridization and wash processing, such as formation of bubbles and incomplete washing.

### BACKGROUND OF THE INVENTION

1. Field of the Invention

The present invention relates to a hybridization apparatus for hybridizing a sample nucleic acid and a probe nucleic acid by using a nucleic acid chip having the probe nucleic acid immobilized thereon.

This application claims priority from Japanese Patent Application No. 2004-021683 filed Jan. 29, 2004, which is hereby incorporated by reference herein.

2. Description of the Related Art

Genetic analysis has recently been performed using a test specimen such as microarray and DNA chip. Under hybridization conditions, a DNA chip having a number of DNA probes arrayed and spotted onto a matrix as probe spots bound on the surface of a substrate made of a slide glass, silicon substrate or the like is brought into contact with a sample such as DNA labeled with fluorochrome. If the detector substance (DNA chip) and the sample contain respective nucleic acids to be hybridized, the labeled substance is fixed onto the detector substance via the probe nucleic acid. The kind of the nucleic acid thus hybridized can be identified by detecting the location of the labeled substance (refer to U.S. Pat. No. 6,476,215, “Ink Jet Method Of Spotting A Probe And Manufacturing A Probe Array”, issued Nov. 5, 2002 to Okamoto, et al.).

FIG. 8 is a schematic view of a conventional hybridization apparatus using a DNA chip. A chamber 12 is placed over a DNA chip 11 and a minute space is disposed between the DNA chip 11 and the chamber 12. When a sample DNA solution is injected onto the DNA chip 11 by a sample DNA solution injector 14, the sample DNA solution diffuses over each DNA spot on the DNA chip 11. After diffusion, the sample DNA solution is heated to a predetermined temperature by a heater 13, whereby hybridization starts. The DNA chip is then washed to remove the substances which have remained unhybridized and therefore unnecessary. After that, hybridization detection is carried out using a hybridization detecting system.

When the hybridization solution does not diffuse uniformly over the whole probe region or bubbles attach to the probe region upon injection of the sample DNA solution onto the DNA chip, however, the hybridization reaction does not occur at some spots, which interferes with production of correct results.

Moreover, during the heating of the hybridization reaction, bubbles sometimes come out of the sample solution. When bubbles appear in the probe region, the hybridization reaction does not occur at some spots, which interferes with production of correct results.

In addition, when a washing liquid does not flow uniformly over the whole chip or foam attaches upon washing after the hybridization reaction, the spots are not washed completely. Such a residue often leads to a background noise upon hybridization detection, which also interferes with production of correct results.

There is accordingly a demand for the provision of a hybridization apparatus capable of reliably performing hybridization reaction by uniformly diffusing a sample DNA solution to probe DNA spots and completely washing the DNA chip with a washing liquid and thereby performing an assay with high precision.

### SUMMARY OF THE INVENTION

The present invention provides a hybridization apparatus for causing hybridization reaction between a sample nucleic acid and a probe nucleic acid by using a nucleic acid chip having the probe nucleic acid bound on the surface of a carrier, which comprises a feeding unit for feeding a liquid to the surface of the carrier and an observing unit for observing the state of the liquid on the surface of the carrier. The hybridization reaction is effected using a chamber member which forms a space for feeding the liquid to the surface of the carrier, and the feeding unit may be an injecting unit for injecting the liquid into the space via an inlet disposed in the chamber member.

Because the state of the liquid is observed, accuracy of the assay is improved since it is possible to detect some of the aforementioned difficulties encountered during hybridization of wash processing.

The observing unit may be a liquid detecting unit for detecting the existing state of the thus-fed liquid as a two-dimensional image.

The hybridization apparatus may be equipped with a hybridization detecting unit for detecting the hybridization reaction and the above-described liquid detecting unit may also serve as the hybridization detecting unit.

The detection by the liquid detecting unit or the hybridization detecting unit may be performed on a side opposite to the surface of the probe carrier having the probe nucleic acid bound thereon.

The hybridization apparatus may further comprise a judging unit for judging the diffusion state of the sample solution based on the detection results by the liquid detecting unit.

The hybridization apparatus may further comprise a control unit for treating the judgment results of the diffusion state of the sample solution and the hybridization detection results by the hybridization detecting unit.

The liquid may be a sample solution containing the sample nucleic acid or a washing solution for washing the space after completion of the hybridization reaction.

The state of the surface of the nucleic acid chip after washing may be assessed based on the detection results by the detecting unit.

The hybridization apparatus may have a heating unit for heating the liquid fed to the surface of the carrier.

The hybridization apparatus may have a stirring unit for stirring the liquid fed to the surface of the carrier.

The present invention also provides a hybridization apparatus for causing hybridization reaction between a sample nucleic acid and a probe nucleic acid by using a nucleic acid chip having the probe nudleic acid bound on the surface of a carrier, which comprises a liquid feeding unit for feeding a liquid to the surface of the carrier and an observing unit for observing the state of the thus-fed liquid.

The hybridization reaction is performed using a chamber member forming a space for feeding the liquid to the surface of the carrier, and the feeding unit may have a tip designed to permit communication with an inlet disposed in the chamber member.

The present invention also provides a method for causing hybridization reaction between a sample nucleic acid and a probe nucleic acid by using a nucleic acid chip having the probe nucleic acid bound on the surface of a carrier, which comprises feeding a liquid to the surface of the carrier and detecting the existing state of the liquid thus fed.

The detection of the existing state of the liquid may be acquisition of the diffusion state of the liquid on the surface of the carrier as a two-dimensional image.

A solution may be fed to give a uniform dispersion state based on the two-dimensional image thus acquired.

The two-dimensional image may be obtained upon feeding a hybridization solution, upon feeding a washing liquid or from a solution under the hybridization reaction conditions.

The solution may be stirred based on the two-dimensional image thus acquired.

The present invention also provides a nucleic acid detecting method for detecting a sample nucleic acid by using a nucleic acid chip having a probe nucleic acid bound onto the surface of a carrier, which comprises a step of performing the above-described hybridization reaction and a step of detecting the sample nucleic acid bound to the probe nucleic acid on the carrier.

The hybridization apparatus of the present invention is able to detect the dispersion state of a sample solution injected onto a nucleic acid chip prior to the hybridization reaction so that it is possible to accurately control the diffusion state of the solution. This makes it possible to reliably control the hybridization reaction between the spotted probe nucleic acid and the sample nucleic acid, resulting in assay with high precision.

In addition, washing can be carried out sufficiently upon washing because the flow of the washing liquid can be detected upon washing. This makes it possible to lessen the influence of this source of background noise, resulting in assay with good precision.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constituted a part of the specification, illustrate embodiments of the invention and, together with the description, serve to explain the principles of the invention.

FIG. 1 is a schematic view of a hybridization apparatus according to a first embodiment.

FIGS. 2A and 2B are schematic views of other constitution example of the hybridization apparatus according to the first embodiment.

FIGS. 3A and 3B are schematic views of other constitution example of the hybridization reaction chamber according to the first embodiment.

FIGS. 4A, 4B and 4C are representative schematic views of the diffusion of a sample solution according to the first embodiment.

FIGS. 5A, 5B and 5C are flow charts of the injection of the sample solution according to the first embodiment.

FIG. 6 is a schematic view of a hybridization apparatus according to a second embodiment.

FIGS. 7A and 7B are schematic views illustrating time-wise sequential diffusion of a sample solution according to the second embodiment.

FIG. 8 is a schematic diagram illustrating the conventional hybridization.

### DETAILED DESCRIPTION OF THE PREFERRED EMBODIMENTS

Preferred embodiments of the present invention will now be described in detail in accordance with the accompanying drawings.

### First Embodiment

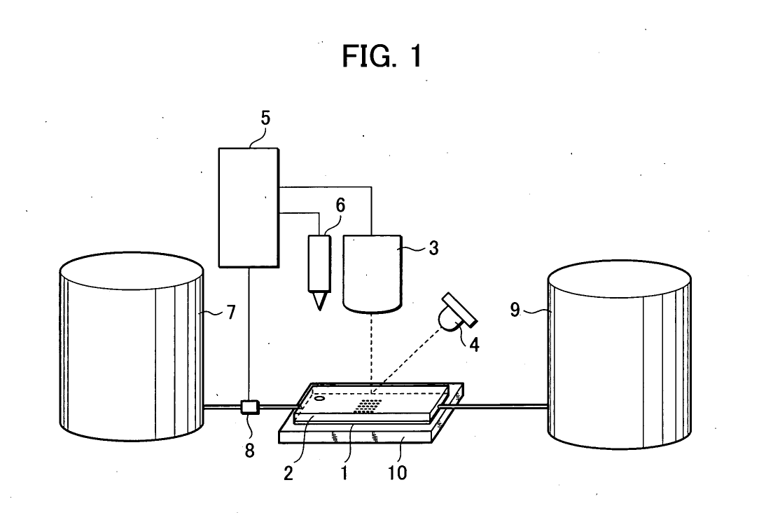

The first embodiment of the present invention will next be described referring to FIGS. 1 to 3.

FIG. 1 illustrates the constitution of the hybridization apparatus according to the present invention. Over a DNA chip 1 having DNA spots, a chamber 2 made of a transparent member is disposed. A minute space is provided between the DNA chip 1 and the chamber 2. Over the chamber, a detecting unit 3 composed of CCD, lens and the like and a light source 4 illuminating the DNA chip are disposed. The output of the detecting unit 3 is connected to a control unit 5. An injecting unit 6 for injecting a sample solution to the DNA chip 1 is connected to the control unit 5. Between a washing liquid tank 7 and DNA chip, a controllable electromagnetic valve 8 is installed and is connected to the control unit 5. On the opposite side of the washing liquid tank 7, a waste water tank 9 is disposed so as to be connected to the DNA chip. Below the DNA chip 1, a heater 10 is disposed in control of control unit 5.

In such a constitution, the light source 4 illuminates the DNA chip 1 through the chamber 2. When the sample solution is injected onto the DNA chip 1 from the injecting unit 6, the detecting unit 3 detects the state of the hybridization solution on the DNA chip 1 and sends the detection results to the control unit 5. The control unit 5 assesses the state of the sample solution. When it assesses that the diffusion is completed, the heater 10 is driven to initiate hybridization reaction. Even during the hybridization reaction, the state of the hybridization solution is observed and its detection results are sent to the control unit 5. The control unit 5 continues the hybridization reaction when it recognizes that nothing abnormal is detected from the state of the hybridization solution. After completion of the hybridization reaction by the heating for a predetermined time, the control unit 5 opens a valve 8 to run a washing liquid over the DNA chip 1. The detecting unit 3 detects the state of the washing liquid spread over the DNA chip 1 at this time and sends the detection results to the control unit 5. The control unit 5 assesses the state of the washing liquid and terminates the washing.

When the detecting unit 3 is composed of an image sensing device such as CCD (charge coupled device), it is possible to detect, by this detecting unit, the hybridization results after completion of the washing.

For example, when the sample DNA is labeled with a fluorescent substance, the fluorescent substance generates fluorescence by the irradiation of a light having a predetermined wavelength onto the DNA chip from a light source not illustrated in FIG. 1. It is also possible to obtain the hybridization results by detecting this fluorescence at the detecting unit 3. If the light source 4 can emit a light having a wavelength capable of exciting a fluorescent substance, use of the light source 4 for detecting the fluorescence can of course be recommended.

In the case of chemiluminescence detection of the sample DNA, on the other hand, it is also possible to adopt a constitution permitting detection of the hybridization results by performing hybridization of the sample DNA labeled in advance with an enzyme, injecting a luminescent reagent after completion of the washing, causing the luminescent reagent to react with the enzyme to generate chemiluminescence, and detecting this light at the detecting unit 3.

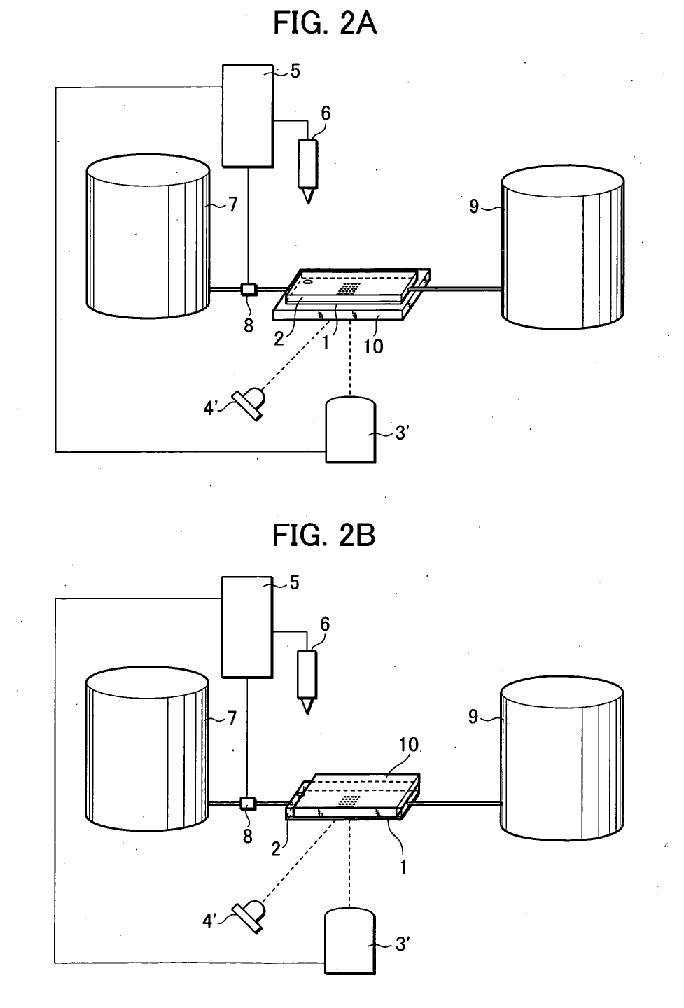

In the detecting method of the above-described embodiment, a detecting unit 3′ and a light source 4′ may be disposed below the DNA chip as illustrated in FIGS. 2A and 2B. When the DNA chip is made of a transparent base material such as slide glass, the light source 4′ illuminates the surface of the DNA chip 1 and the space between the DNA chip 1 and the chamber 2 through the base material of the DNA chip 1, whereby the state of the sample solution or hybridization results can be detected by the detecting unit 3′.

In FIG. 2A, when the detection is performed by the detecting unit 3′, the heater 10 is transferred to a position not affecting the detection. As illustrated in FIG. 2B, the heater 10 may be disposed over the chamber 2 and the sample solution may be heated from the upper surface of the chamber 2.

The image is preferably obtained as a moving image such as video image or successive images at intervals of certain periods. The diffusion state of the liquid can be found from a change in the image.

A substance for enhancing the liquid detection sensitivity, for example, a substance for coloring the sample solution may be mixed in the solution to assess the dispersion state more correctly.

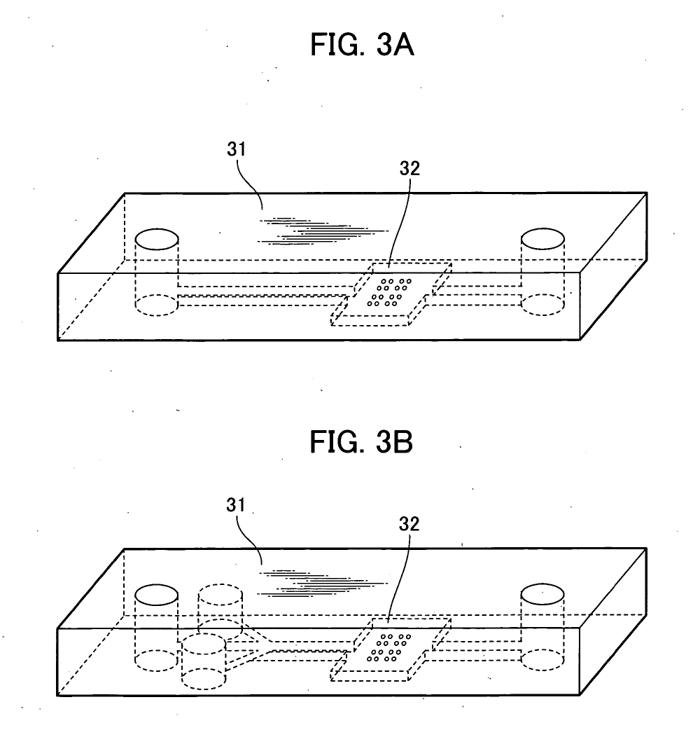

In these diagrams, the chamber 2 is separated from the DNA chip 1, but they may be integrated insofar as a space for hybridization is disposed. For example, as illustrated in FIGS. 3A and 3B, a box 31 having a hybridization reaction chamber portion 32 may be employed, connected by one or more injection inlets.

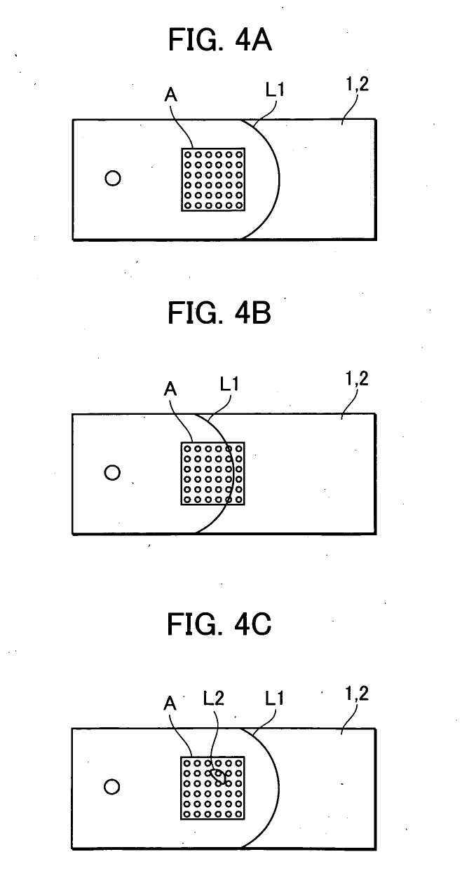

FIGS. 4A, 4B and 4C are representative images detected by the detecting unit 3, in which A is a frame encompassing a DNA spot region, L1 is the end of a sample solution and L2 is a bubble in the sample solution. FIG. 4A is a diagram illustrating the appropriate diffusion state. The sample solution L1 spreads to cover the spot region A of the probe DNA. In this diagram, the sample solution is ready to react with all the DNA spots. FIG. 4B is a diagram illustrating an insufficient diffusion state, in which the sample solution L1 partially exists inside of the DNA spot region A and does not spread all over the DNA spot region. FIG. 4C is a diagram illustrating a bubble generated in the sample solution. The bubble L2 exists inside of the DNA spot region. In the case of 4B or 4C, some DNA spots cannot undergo hybridization reaction. The diffusion state is detected by comparing and assessing a positional relationship among the DNA spot region A, sample solution L1 and bubble L2, which is available from these images, by the control unit 5. The state of the hybridization solution during heating or the state of the washing liquid upon washing can be detected by the above-described assessing method.

Although the frame A showing the spot region of the probe DNA is indicated on the DNA chip 1, it may be indicated so that the detecting unit 3 can include the whole DNA spot region. For example, it may be indicated on a cover member or, when the DNA chip is placed on another plate, it may be indicated on the plate. Any form may be adopted for indicating the frame A insofar as it permits the recognition by the control unit 5. For example, the contour may be indicated with a continuous line or a broken line or by arrangement of plural dots; the whole contour of the substrate of the DNA chip may be recognized as the spot region; or spots themselves may be recognized. It is also possible to dispose another flow sensor in the flow channel of the washing liquid and carry out assessment in combination with the flow sensor.

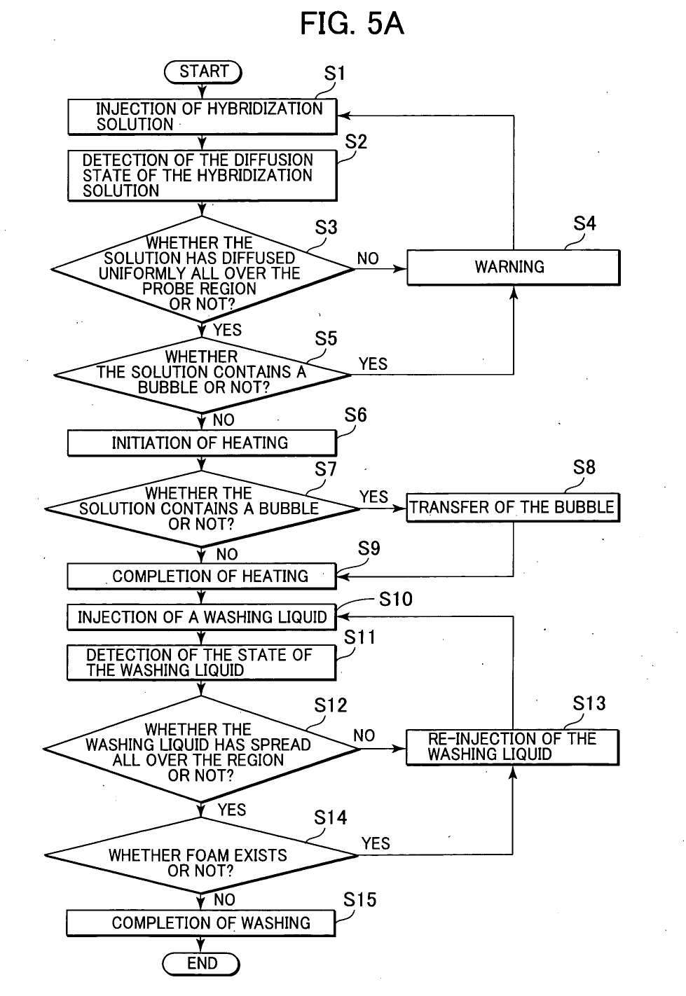

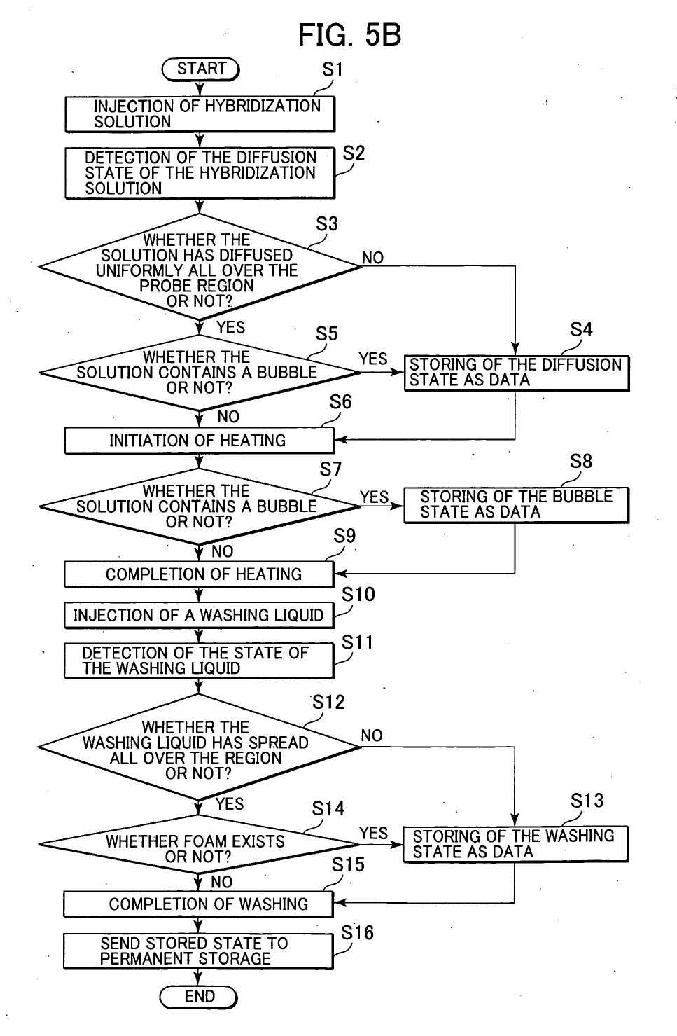

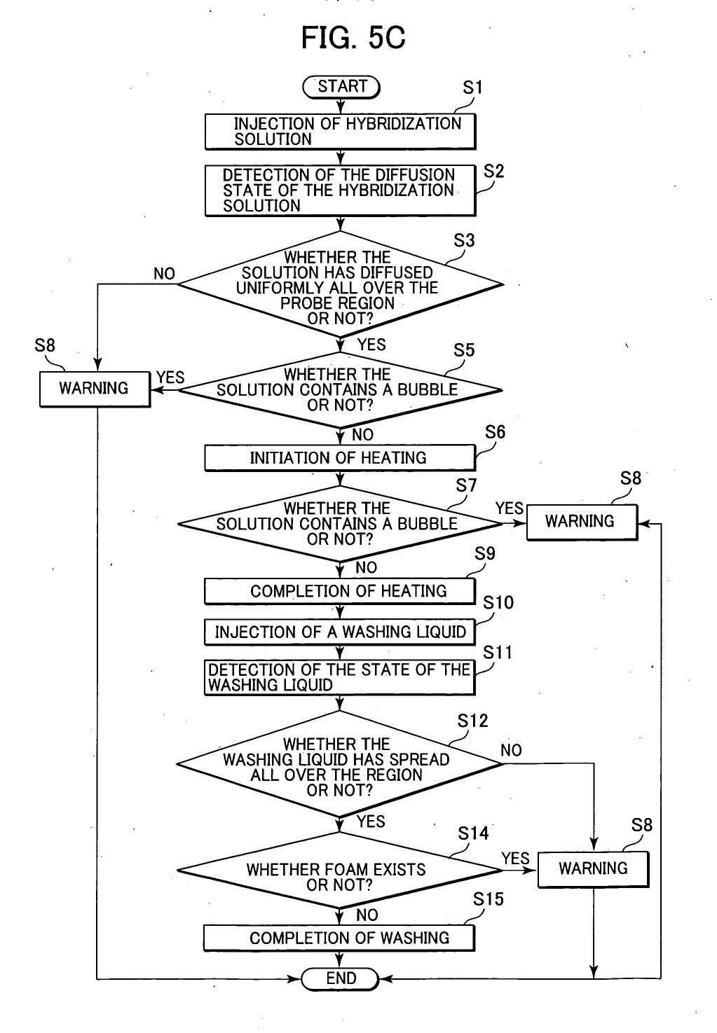

FIGS. 5A, 5B and 5C are flow charts of this embodiment.

In FIG. 5A, the sample solution injected from the injecting unit 6 in Step S1 diffuses over the DNA chip 1. In Step S2, the diffusion state of the sample solution is detected, and in Step S3, whether the solution spreads all over the probe region or not is determined. When the diffusion state of the solution is insufficient, a warning is given in Step S4 and injection of the sample solution in Step S1 is performed again. When the diffusion state of the solution is sufficient, on the other hand, presence or absence of a bubble in the sample solution is determined in Step S5. In the case of presence of a bubble, a warning is given in Step S4 and injection of the sample solution in Step 1 is performed again. In the case of absence of a bubble, heating by the heater 10 is started in Step S6. In Step S7, presence or absence of a bubble in the sample solution is determined. In the case of presence of a bubble, the transfer of the bubble is performed in Step S8, and after heating for a predetermined time, heating is terminated in Step S9. When the solution does not contain any bubble, on the other hand, heating is performed for the predetermined time and then, heating is terminated in Step S9. In Step S10, a washing liquid is injected. In Step S11, the state of the washing liquid is detected. In Step S12, whether the washing liquid flows uniformly all over the region or not is determined. When the liquid flows only partially, warning is given in Step S13 and injection of the washing liquid is continued again in Step S10. When the washing liquid flows uniformly all over the region, on the other hand, the presence or absence of foam is determined in Step S14. In the case of presence of foam, a warning is given in Step S13 and injection of the washing liquid in Step S10 is performed again. When the solution does not contain foam, on the other hand, the washing is terminated in Step S15.

In Step S14, the presence of foam means (for example) that foam stays at one position for a predetermined time, while the absence of foam means that it does not.

The warning in Step S5 may be given by any method capable of alerting a person in charge and a sound such as buzzer or caution-advisory indicator may be used. Alternatively, when the apparatus has a constitution to permit display of the observation image at the detecting unit on a monitor, the warning may be indicated on the monitor.

In this embodiment, the warning is given in Step S4 in accordance with the judgment results of Steps S3 and S5, but it is possible to repeat Step S1 automatically, even without any warning at all.

In this Embodiment, the sample solution is injected again when the diffusion state is insufficient. Any diffusing method can be employed insofar as it can sufficiently diffuse the sample solution. The sample solution can be diffused not by injection but, for example, by pressurization or tilting of the DNA chip.

In Step S8, the transfer of the bubble is performed. The bubble can be transferred, for example, by disposing an oscillation unit such as ultrasonic transducer in the vicinity of the chamber or by repeating injection and discharge of the solution, thereby stirring it.

The detection of the bubble is conducted in Step S7 and based on the detection results, the transfer of the bubble is performed in Step S8. Alternatively, the persistence of the bubble, if any, at a certain place may be prevented by actuating the above-described oscillation unit or applying the above-described injection/discharge method continuously irrespective of the detection results in Step S7.

This makes it possible to deal with the generation of bubbles in a short span of time so that the hybridization reaction on the surface of the carrier becomes more uniform.

FIG. 5B is a flow chart of another treatment when the judgment in each of the steps S3, S5, S7, S12 and S14 in FIG. 5A is adverse. When the judgment in the above-described steps is adverse, the state of the solution or liquid is stored in the control unit 5, followed by the next step. After the completion of Step S15, the state of the solution or liquid thus stored is sent to a hybridization reaction detecting apparatus (Step S16) via a recording medium such as floppy (trade mark) disk or via a network. The results of the hybridization reaction and the state of the solution or liquid thus sent to the apparatus are compared and unreliable data are neglected or deleted.

The hybridization apparatus can compare data readily if it has a function of detecting hybridization reaction.

FIG. 5C is a flow chart of a further treatment to be employed when the judgment in each of Steps S3, S5, S7, S12 and S14 in FIG. 5A is adverse. When the judgment is adverse, warning is given in Step S8, followed by terminating treatment of hybridization.

### Second Embodiment

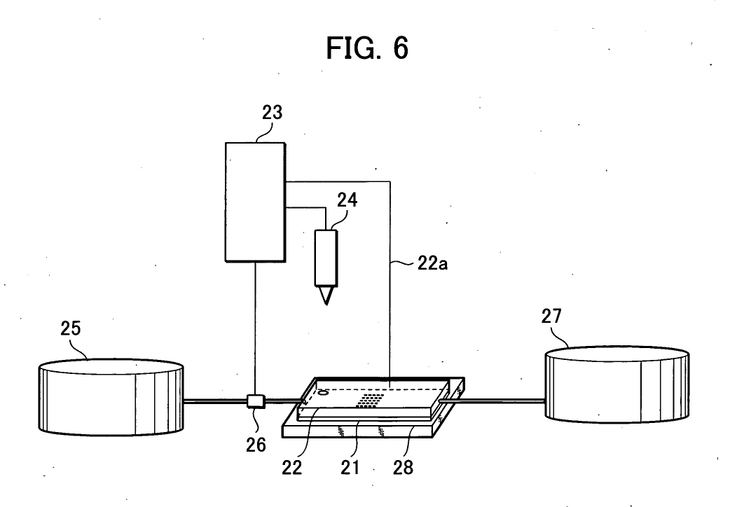

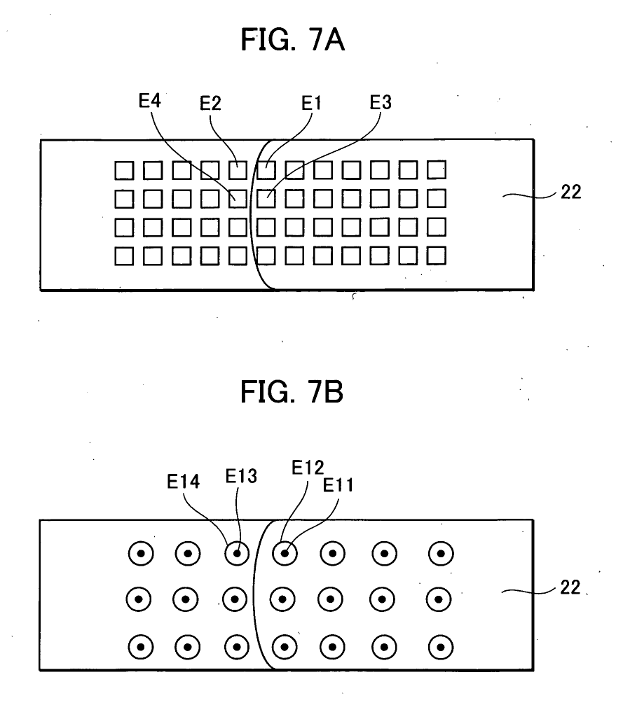

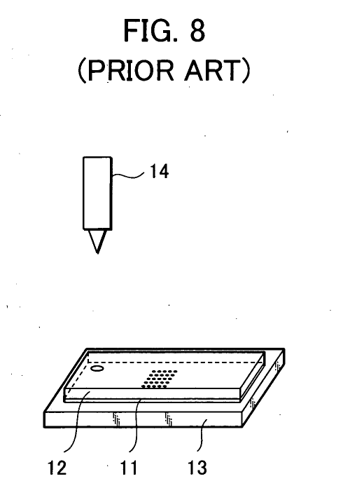

A description will next be made of the second embodiment referring to FIGS. 6 and 7.

FIG. 6 illustrates the constitution of the hybridization apparatus according to the present invention. Over a DNA chip 21 having DNA spots, a chamber 22 having a liquid detecting electrode (see FIGS. 7A and 7B) is disposed. A minute space is provided between the DNA chip 21 and the chamber 22. The output 22a of the liquid detecting unit of the chamber 22 is connected to a control unit 23. An injecting unit 24 for injecting a sample solution to the DNA chip 21 is also connected to the control unit 23. Between a washing liquid tank 25 and the DNA chip, a controllable electromagnetic valve 26 is disposed and it is connected to the control unit 23. On the opposite side of the washing liquid tank 25, a waste liquid tank 27 is disposed in connection with the DNA chip 21. Below the DNA chip 21, a heater 28 is disposed in connection with the control unit 23.

In such a constitution, when the sample solution is injected onto the DNA chip 21 from the injecting unit 24, the liquid detecting electrode of chamber 22 detects the state of the sample solution on the DNA chip 21 and sends the detection results to the control unit 23. When the control unit 23 assesses, based on the state of the sample solution, that the diffusion is finished, heating is conducted using the heater 28 to initiate the hybridization reaction. When the hybridization reaction is completed by heating for a predetermined time, the control unit 23 opens the valve 26 and runs the washing liquid over the DNA chip 21. The liquid detecting electrode of chamber 22 at this time detects the state of the washing liquid over the DNA chip 21 and sends the detection results to the control unit 23. The control unit 23 assesses the state of the washing liquid and terminates the washing.

FIGS. 7A and 7B are each a schematic view of the chamber 22.

In FIG. 7A, a plurality of electrodes E1, E2, etc. are formed on the surface opposite to the substrate 21 of the chamber 22 and a voltage is applied so that any two adjacent electrodes have alternately positive and negative potentials, respectively.

As liquid begins to flow in chamber 22, the electrodes E1 and E3 are contacted with the solution, while the electrodes E2 and E4 are contacted with not the solution but air. Since the solution and air are different in conductivity, a range within which the solution has reached can be found by detecting the value of current or resistance, for example, between E1 and E2, E1 and E3, E2 and E4 or E3 and E4.

In FIG. 7B, electrodes E12 and E14 are disposed to surround therewith electrodes E11 and E13, respectively. A voltage is applied to E11 and E12 so that they have alternately positive and negative potentials, respectively. This also applies to E13 and E14. A range within which the solution has reached can be found by detecting the resistance between E11 and E12 and that between E13 and E14.

In this embodiment, the electrodes are disposed in the chamber 22, but they may be disposed on the side of the substrate 21.

Although not illustrated in the above-described embodiments, a step for drying the washing liquid can be added after the washing step. It is needless to say that the dried state can be detected.

In the embodiments of the present invention, a DNA chip having a probe DNA bound thereon is used, but the hybridization apparatus can be applied to not only to it but also to a chip having, fixed thereon, a nucleic acid to be subjected to hybridization such as RNA, cDNA (complementary DNA), PNA, oligonucleotide or polynucleotide.

The present invention is not limited to the above embodiments and various changes and modifications can be made within the spirit and scope of the present invention. Therefore to apprise the public of the scope of the present invention, the following claims are made.

1. A hybridization apparatus for causing hybridization reaction between a sample nucleic acid and a probe nucleic acid by using a nucleic acid chip having the probe nucleic acid bound on the surface of a carrier, which comprises:
a feeding unit for feeding a liquid to the surface of the carrier, and an observing unit for observing the state of the liquid on the surface of the carrier.

2. A hybridization apparatus according to claim 1, wherein the hybridization reaction is performed using a chamber member which forms a space for feeding the liquid to the surface of the carrier, and the feeding unit is an injecting unit for injecting the liquid to the space via an inlet disposed in the chamber member.

3. A hybridization apparatus according to claim 2, wherein the observing unit is a liquid detecting unit for detecting, as a two-dimensional image, the existing state of the liquid fed to the surface.

4. A hybridization apparatus according to claim 3, further comprising a hybridization detecting unit for detecting the hybridization reaction.

5. A hybridization apparatus according to claim 4, wherein the liquid detecting unit also serves as the hybridization detecting unit.

6. A hybridization apparatus according to claim 4, wherein the detection by the liquid detecting unit or the hybridization detecting unit is performed from a side opposite to the surface of the probe carrier on which the probe nucleic acid is bound.

7. A hybridization apparatus according to claim 4, wherein the liquid comprises a sample solution of the sample nucleic acid, and further comprising a judging unit for judging the dispersion state of the sample solution based on the detection results by the liquid detecting unit.

8. A hybridization apparatus according to claim 4, further comprising a control unit for controlling in accordance with results of the judgment on the dispersion state of the sample solution and hybridization detection results by the hybridization detecting unit.

9. A hybridization apparatus according to claim 1, wherein the liquid is a sample solution containing the sample nucleic acid.

10. A hybridization apparatus according to claim 1, wherein the liquid is a washing liquid for washing the space after the hybridization reaction.

11. A hybridization apparatus according to claim 10, wherein the washed state on the surface of the nucleic acid chip is assessed based on the liquid state observed by the observation unit.

12. A hybridization apparatus according to claim 1, further comprising a heating unit for heating the liquid fed to the surface of the carrier.

13. A hybridization apparatus according to claim 1, further comprising a stirring unit for stirring the liquid fed to the surface of the carrier.

14. A method for causing hybridization reaction between a sample nucleic acid and a probe nucleic acid by using a nucleic acid chip having the probe nucleic acid bound on the surface of a carrier, which comprises:
feeding a liquid to the surface of the carrier and detecting the existing state of the liquid thus fed to the surface.

15. A method according to claim 14, wherein the detection of the existing state of the liquid is to obtain the diffusion state of the liquid on the surface of the carrier as a two-dimensional image.

16. A method according to claim 15, wherein the liquid is fed to attain a uniform diffusion state in accordance with the two-dimensional image thus obtained.

17. A method according to claim 15, wherein the liquid comprises a hybridization solution of the sample nucleic acid, and wherein the two-dimensional image is obtained upon feeding of the hybridization solution.

18. A method according to claim 15, wherein the liquid comprises a washing liquid, and wherein the two-dimensional image is obtained upon feeding of the washing liquid.

19. A method according to claim 15, wherein the liquid comprises a hybridization solution of the sample nucleic acid, and wherein the two-dimensional image of the hybridization solution is obtained during hybridizaiton.

20. A method according to claim 15, wherein the solution is stirred in accordance with the two-dimensional image obtained.

21. A detecting method of a sample nucleic acid by using a nucleic acid chip having a probe nucleic acid bound on the surface of a carrier, which comprises:
effecting hybridization reaction as claimed in any one of claims 15 to 20, and
detecting the sample nucleic acid bound with the probe nucleic acid on the carrier.
